(19) United States
(12) Patent Application Publication
(10) Pub. No.: US 2004/0010174 A1
(43) Pub. Date: Jan. 15, 2004

(54) Oxidative dehydrogenation of hydrocarbons by promoted metal oxides

(75) Inventors: Daxiang Wang, Ponca City, OK (US); Joe D. Allison, Ponca City, OK (US)

Correspondence Address: DAVID W. WESTPHAL, CONOCOPHILLIPS COMPNAY, P.O. BOX 1267, PONCA CITY, OK, 74602-1267, US

(73) Assignee: Conoco Inc., Houston, TX

(21) Appl. No.: 10/195,222

(22) Filed: Jul. 15, 2002

(51) Int. Cl. C07C 5/333; B01J 23/10

(52) U.S. Cl. 585/658; 502/302; 585/661

(57) ABSTRACT

A catalyst system and process for use in ODH that allows high conversion of hydrocarbon feedstock at high gas velocities, while maintaining high selectivity of the process to the desired products. In accordance with a preferred embodiment, a catalyst for use in ODH processes includes a dehydrogenative catalytically active component and an oxidative catalytically active component. The catalyst preferably has the general formula αAOₓ-βBO_{y}-γCO_{z}, wherein A is a precious metal and/or transition metal, B is a rare earth metal, C is an element chosen from Groups IIA, IIIA, and IVA, and O is oxygen. In accordance with another preferred embodiment, a method for converting gaseous hydrocarbons to olefins includes reacting an alkane feed stream with an oxidized bifunctional catalyst in a riser reactor to produce product vapors containing olefins and paraffins and a reduced catalyst.

### FIELD OF THE INVENTION

This invention relates to an oxidative dehydrogenation catalyst composition and a method of using such catalysts in the presence of hydrocarbons. More particularly this invention relates to the compositions of bifunctional catalysts for the production of olefins by oxidative dehydrogenation of hydrocarbons in a circulating fluidized bed (CFB) reactor/regenerator system.

### BACKGROUND OF THE INVENTION

Dehydrogenation of hydrocarbons is an important commercial process. Dehydrogenation is the process used to convert aliphatics to olefins, mono-olefins to di-olefins, cycloalkanes to aromatics, alcohols to aldehydes and ketones, aliphatics and olefins to oxygenates, etc., by removing hydrogen chemically. In more practical terms, this process is responsible for products such as detergents, gasolines, pharmaceuticals, plastics, polymers, synthetic rubbers and many others. In addition, there is significant commercial use of the process for making many of the precursors for the above mentioned products. For example, polyethylene is made from ethylene, which is made from the dehydrogenation of ethane (i.e. aliphatic to olefin). More ethylene is produced in the U.S. than any other organic chemical. Thus, it is easy to appreciate the significance of the dehydrogenation process to industry.

Light olefins are mainly produced as byproducts from fluidized catalytic cracking (FCC) and/or steam pyrolysis in the production of ethylene. Commercial interest in propane dehydrogenation has been increasing and numerous research efforts have been attempted in the development of related catalysts and processes. An obvious advantage of catalytic dehydrogenation is its low operating temperature (e.g. 400-500° C.), compared to the high operating temperature of steam cracking (e.g. 800-1000° C.).

The dehydrogenation of paraffins is a highly endothermic reaction. Endothermic reactions absorb heat. The heat necessary to drive the reaction can be provided to the reactor through various methods. One method includes heating the reactor with a heat exchanger, similar to those used in pyrolysis furnaces. Another method includes adding hot steam to the paraffin feedstock, allowing the feedstock to act as a heat carrier providing the reaction heat.

In addition, the dehydrogenation reaction is an equilibrium-limited reaction. Table 1 shows the equilibrium yield of several olefins from the dehydrogenation of related paraffins (i.e. propane→propylene, butane→butylene).

TABLE 1

| Temperature | Propane | n-Butane | i-Butane |
|---|---|---|---|
| (° C.) | (mol %) | (mol %) | (mol %) |
| 350 | 2 | 3 | 4 |
| 400 | 4 | 7 | 8 |
| 450 | 9 | 15 | 18 |
| 500 | 18 | 28 | 33 |
| 550 | 32 | 46 | 53 |
| 600 | 50 | 66 | 72 |
| 650 | 68 | 82 | 85 |
| 700 | 82 | 92 | 93 |

At the temperature range of interest for catalytic dehydrogenation (400-500° C.), the equilibrium yields are too low to have substantial commercial significance. One strategy to overcome the thermodynamic limit is to remove one of the products out of the reaction system (i.e. hydrogen) so as to shift the equilibrium. To date, membrane reactors have been examined for this purpose. However, due to the low permittivity, or the poor selectivity of available membranes, membrane reactors for the dehydrogenation of paraffins are still in the preliminary development stage. An alternative technique for removing hydrogen is to employ oxidative dehydrogenation (ODH). This strategy is attracting increased interest and many catalyst systems, such as supported molten salt catalysts based on alkali chlorides, lithium hydroxide/lithium iodide-melt catalysts, metal sulfide catalysts, metal phosphate catalysts, nickel molybdenate catalysts, niobium pentoxide catalysts, and vanadium-magnesium catalysts have been developed for this use. The common feature of these catalytic systems is their low selectivity to the formation of olefins, due to the relatively high reactivity of the dehydrogenation product in the existence of gas phase oxygen.

Despite a vast amount of research effort in this field, there is still a great need to identify effective catalyst systems for olefin synthesis, so as to maximize the value of the olefins produced and thus maximize the process economics. In addition, to ensure successful operation on a commercial scale, the ODH process must be able to achieve a high conversion of the hydrocarbon feedstock at high gas velocities (compared to fixed beds), while maintaining high selectivity of the process to the desired products.

### SUMMARY OF THE INVENTION

The present invention provides a catalyst system and process for use in ODH that allow high conversion of the hydrocarbon feedstock at high gas velocities, while maintaining high selectivity of the process to the desired products. For the purposes of this disclosure, all listed metals are identified using the CAS naming convention.

In accordance with a preferred embodiment of the present invention, a catalyst for use in ODH processes includes a dehydrogenative catalytically active component and an oxidative catalytically active component. The catalyst preferably has the general formula αAOₓ-βBO_{y}-γCO_{z}, wherein A is a precious metal and/or transition metal, B is a rare earth metal, C is an element chosen from Groups IIA, IIIA, and IVA, and O is oxygen.

In accordance with another preferred embodiment of the present invention, a method for converting gaseous hydrocarbons to olefins includes reacting an alkane feed stream with an oxidized bifunctional catalyst in a riser reactor to produce product vapors containing olefins and paraffins and a reduced catalyst.

The combination of the dehydrogenation and oxidation processes results in a more efficient, lower cost olefin process.

### BRIEF DESCRIPTION OF THE DRAWING

For a more detailed understanding of the present invention, reference is made to the accompanying Figure, which is a schematic diagram of a reactor system constructed in accordance with a preferred embodiment of the invention.

### DETAILED DESCRIPTION OF THE PREFERRED EMBODIMENTS

According to a preferred embodiment, two catalyst components are combined to form one ODH catalyst system for converting alkanes to olefins. In a preferred embodiment of the present invention, light alkanes and oxygen are converted to the corresponding olefins using novel bifunctional catalysts. The oxygen may be lattice oxygen and/or absorbed oxygen. Lattice oxygen is herein defined as those oxygen ions coordinated to metallic cations in the bulk of the metal oxide, while absorbed oxygen is defined as those oxygen species adsorbed on the catalyst surface as molecular oxygen O₂, and/or ionic oxygen such as O⁻, O₂²⁻, O²⁻, etc.

Catalysts

A preferred embodiment of the present invention comprises using novel, highly active and selective metal oxide supported metal/metal oxide catalysts to carry out oxidative dehydrogenation. The preferred catalysts are bifunctional. In a first function, the catalyst preferably possesses a high dehydrogenation activity, allowing it to quickly reach the equilibrium of the dehydrogenation reaction. In a second function, the catalyst preferably possesses a sufficient oxidative ability to convert the produced hydrogen in situ so as to drive the dehydrogenation equilibrium to favor olefin production.

These catalysts preferably have the general formula αAOₓ-PBO_{y}-γCO_{z}, wherein:

A is one of the precious metals Rh, Ru, Pd, Pt, Au, Ag, Os or Ir or is a transition metal chosen from the group consisting of Sc, Ti, V, Cr, Mm, Fe, Co, Ni, Cu, Zn, Nb, Mo, Tc, Ru, Rh, Pd, Ag, Hf, Ta, W, Re, preferably Pt, Au, Ag, Fe, Co, Ni, Mn, V or Mo or any combination thereof;

B is a rare earth metal La, Ce, Pr, Nd, Pm, Sm, Eu, Gd, Th, Dy, Ho, Er, Tm, Yb, Lu, Sc, Y and Th, preferably La, Yb, Sm or Ce;

C is an element chosen from Group IIA (i.e., Be, Mg, Ca, Sr, Ba and Ra), IIIA (i.e., B, Al, Ga, In, Ti) and IVA (i.e., C, Si, Ge, Sn, Pb), preferably Mg, Al or Si;

O is oxygen;

α, β, γ are the relative molar ratios of each metal oxide and α=0-0.2; β=0-0.5; γ=0.5-1; and

x, y, z are the numbers determined by the valence requirements of the metals A, B, and C, respectively. Their value can be zero when the corresponding metal stays in the metallic state.

In the above general formula, components CO_{z} can be made of zeolite (also called as molecular sieves). In this general formula, if component A is in metallic form, this general formula can be presented as αA-βBO_{y}-γCO_{z}.

In a preferred embodiment, the catalyst may or may not be supported. In an unsupported catalyst or mixed phase catalyst, the catalyst is preferably formed by the combination of two separately optimized catalyst systems for dehydrogenation and oxidation. The criterion for an oxidation catalyst include (i) that the catalyst be active for the oxidation of hydrogen and (ii) that the catalyst be inert for the combustion of alkanes and the produced olefins. In a preferred embodiment, the catalysts for the two individual reactions are first optimized and are then combined through extrusion or compressing processes with bindery materials. The balance of the two functions may be manipulated by adjusting the ratio of the two catalyst components.

The advantages of this catalyst design include (i) successful formulas are available from open literature on catalysts for dehydrogenation and oxidation reactions (ii) direct combination of two systems may balance the two reactions in the ODH reactor and (iii) a kinetic study on separately optimized catalyst systems and the combined catalysts may provide an understanding of the catalytic chemistry of ODH.

In a supported catalyst, the catalyst is preferably formed by the combination of supported metals and metal oxides. It is highly desirable that the support materials possess sufficient attrition resistance for use in the circulating operation.

Catalyst Preparation

The preferred catalysts of the present invention can be prepared through any impregnation and co-precipitation techniques known in the art. Impregnation techniques are more preferred, especially when noble metals such as Pt and/or Au are used.

When the catalysts are prepared by impregnation, a support material must be selected. The support material should have a high surface area and a wide variety of pore structures. Although many support materials are suitable, the preferred support material is selected from the group comprising alumina, silica, titania, magnesia, zirconia, silicon carbide, active carbon mixture thereof. After selecting a support material, a liquid solution containing the active metal components is impregnated onto the support using either the incipient wetness technique or by soaking the support in excess solution. The solid material is then dried starting at room temperature and then ramped up to around 120° C. The resulting catalyst material is then calcined at 200 to 800° C. to decompose the precursor compound(s) into their corresponding metal oxides.

When multi-components are used, such as those expressed in the formula of αAOₓ-βPBO_{y}-βCO_{z}, stepwise or co-impregnation can be used. Stepwise impregnation is performed by impregnating one component, as described above, followed by the impregnation of the next component. Calcination in between the impregnation of each component is optional depending on the exact metals used. Alternatively, a co-impregnation method can be used in preparing multi-components catalysts. In this method, a mixed solution containing all desired metal elements is impregnated onto the catalyst support material in one step followed by drying and calcination.

Some of the preferred catalysts will be active after calcination. However, most catalysts may need to be reduced after calcination to achieve an active catalyst. The temperature range of 200-700° C. to convert the active component from oxide to its metallic state. The examples set out below are representative of catalysts in accordance with a preferred embodiment of the present invention.

The present catalysts are preferably provided in the form of distinct structures. The terms “distinct” or “discrete” structures or particulates, as used herein, refer to supports in the form of divided materials such as granules, beads, pills, pellets, cylinders, trilobes, extrudates, spheres or other rounded shapes, or another manufactured configuration. Preferably at least a majority (i.e., >50%) of the particles or distinct structures have a maximum characteristic length (i.e., longest dimension) of less than three millimeters, preferably less than one millimeters. In a preferred embodiment, the distinct structures have a characteristic length in the range of 0.5 to 1 mm.

Preferably, a circulating fluidized bed (CFB) reactor/regenerator system is used. This technology has the potential to achieve yields above that of the conventional technology at a much lower cost. Additionally, there is minimal coking in the present process and therefore little unit down time and loss of valuable hydrocarbon feedstock. Furthermore, the present novel catalysts improve the selectivity of the process to the desired olefins.

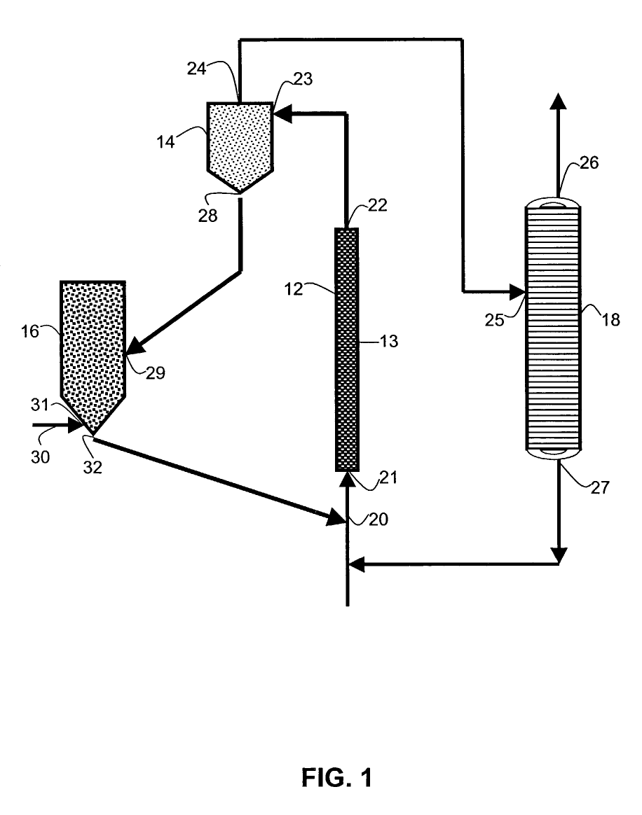

Referring now to FIG. 1, a circulating fluidized bed (CFB) reactor/regenerator system 10 is shown. CFB reactor/regenerator system 10 includes a riser reactor 12, a solid-gas separation vessel 14, a regeneration reactor 16 and a gas-gas separation vessel 18. Initially, an oxidized catalyst (not shown) is loaded into riser reactor 12. A paraffin gas feed 20, preferably enters riser reactor 12 through inlet 21. Here, the endothermic dehydrogenation reaction and the exothermic oxidation reaction occur simultaneously, forming products that include olefins and coke. In riser reactor 12, as the name implies, the catalyst, the feed, and product hydrocarbon mixture rise up reactor pipe 13. At the top of riser reactor 12, the mixture exits via outlet 22 and enters solid-gas separation vessel 14 at inlet 23. In solid-gas separation vessel 14, the catalyst is mechanically separated from the product vapors. The product vapors exit solid-gas separation vessel 14 at outlet 24 and enter gas-gas separation vessel 18 at inlet 25. In the gas-gas separation vessel 18, the product vapors are preferably separated by boiling point differences. Because olefins typically have lower boiling points than paraffins, the olefins are removed at outlet 26, where they are further separated by methods known to those of ordinary skill in the art. The unconverted paraffins are then removed from gas-gas separation vessel 18 at outlet 27 and preferably recycled back into riser reactor 12.

Meanwhile, the coked catalyst that is mechanically separated from the product vapors, exits solid-gas separator 14 at outlet 28 and enters regeneration reactor 16 at inlet 29. An air feed 30 preferably enters regeneration reactor 16 through inlet 31. Here, the combustion of coke on the catalyst (and any hydrocarbons still absorbed that were not removed in the solid-gas separator 14) occurs with the liberation of heat. At the same time, the reduced catalyst is recirculated from riser reactor 12, it is oxidized with oxygen in regeneration reactor 16. This oxidation reaction is also exothermic. Regenerator temperatures are typically 200° C. to 700° C. In a preferred embodiment, the newly oxidized catalyst captures the heat evolved during the regeneration. In another embodiment, the reaction heat can be transferred outside of the regeneration reactor 16 through a heat exchange device (not shown), such as a cooling coil, installed in regeneration reactor 16. The oxidized catalyst exits regeneration reactor 16 at outlet 32 and is recycled back into riser reactor 12.

In a preferred embodiment, a circulating fluidized bed (CFB) reactor/regenerator system, as shown in FIG. 1, is applied to the catalytic oxidative dehydrogenation of paraffins for reasons including heat balance, regeneration realization, and oxygen cost savings.

As is known, the reaction heat for the dehydrogenation of paraffins may be balanced with the exothermic oxidation of hydrogen with oxygen carried by the oxidized catalyst. Manipulation of the oxidation activity and the dehydrogenation activity of the catalyst may result in balanced endothermic and exothermic reactions, making the overall process adiabatic. This is economically desirable because it eliminates the need for additional heating or cooling equipment.

As discussed above, the optimum reaction temperature for the catalytic dehydrogenation of paraffins on various catalysts systems occur in the range of 450-600° C. Within this temperature range, numerous metal oxides with modest redox capacity are very active for the oxidation of hydrogen, but nonactive for the oxidation of olefins and paraffins.

After using the catalyst in the ODH reactor, the reduced catalyst is easily oxidized in air in the temperature range used for ODH. As a result, no heat is necessary for the regeneration of the catalyst. In addition, because the regeneration of the reduced catalyst may be achieved by oxidation in air, as opposed to pure oxygen, the cost of olefin production is decreased. Also, product separation cost is decreased due to the decreased separation load.

In summary, the present catalysts act as oxygen carriers supplying lattice and/or absorbed oxygen for the selective, oxidative dehydrogenation of hydrocarbons in a reduction zone (riser reactor). The reduced catalysts are transferred to a regeneration zone (regeneration reactor), where they are oxidized with air. By circulating the catalysts between these two zones, hydrocarbons are continuously converted to olefins at a stable productivity.

Catalyst Examples

A catalyst comprising Pt and CeO₂ on alumina was prepared through the stepwise impregnation method. Aldrich alumina was selected as the catalyst support. The alumina had a particle size of ca 150 mesh and surface area of 155 square meters per gram. Hydrogen hexachloroplatinate (IV) (H₂PtCl₆)(Aldrich), 8 wt. % solution in water was used as a platinum precursor. Ce(NO₃)₃.6H₂O (Aldrich) was used as a precursor for CeO₂. First, the desired amount of Ce(NO₃)₃.6H₂O was dissolved in de-ionized water and the solution was impregnated on 10 g of alumina to incipient wetness. The sample was then dried at 120° C. for 2 hours and calcined at 400° C. for 2 hours. The material was then impregnated with the desired amount of platinum solution. The impregnated sample was then dried at 120° C. for 12 hours and calcined at 650° C. for 5 hours in flowing air at 50 m./min. Catalyst materials made according to the foregoing steps preferably contain 0.001 to 5 weight percent Pt and 5 to 25 weight percent CeO₂, more preferably 0.01 to 3 weight percent Pt and 10 to 20 weight percent CeO₂, and still more preferably 0.1 to 3 weight percent Pt and 10 to 18 weight percent CeO₂.

Process Conditions

A feed stream comprising a hydrocarbon feedstock is contacted with one of the above-described bifunctional catalysts in a reaction zone maintained at conversion-promoting conditions effective to produce an effluent stream comprising olefins. The hydrocarbon feedstock may be any gaseous hydrocarbon having a low boiling point, such as ethane, natural gas, associated gas, or other sources of light hydrocarbons having from 2 to 10 carbon atoms. In addition, hydrocarbon feeds including naphtha and similar feeds may be employed. The hydrocarbon feedstock may be a gas arising from naturally occurring reserves of ethane, which contain carbon dioxide. Preferably, the feed comprises at least 50% by volume alkanes (<C₁₀).

The process is operated at atmospheric or superatmospheric pressures, the latter being preferred. The pressures may be from about 100 kPa to about 10,000 kPa, preferably from about 100 kPa to about 3,000 kPa. The preheat temperature of the present invention occurs at temperatures of from about 25° C. to about 600° C., preferably from about 150° C. to about 500° C. The preheat temperature is herein defined as the temperature at which the hydrocarbon feedstock is heated up to before entering the riser reactor and contacting the catalyst. The hydrocarbon feedstock is passed over the catalyst at any of a variety of velocities.

The preferred contact time of the hydrocarbon feedstock with a catalyst in the riser reactor is in the range of 0.1-10 seconds. An effluent stream of product gases, including alkenes, CO, CO₂, H₂, H₂O, and unconverted alkanes emerges from the reactor. In some embodiments, unconverted alkanes may be separated from the effluent stream of product gases and recycled back into the feed.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, the present invention may be incorporated into a gas to liquids plant (GTL) or may stand alone. Accordingly, the scope of protection is not limited by the description set out above, but is only limited by the claims which follow, that scope including all equivalents of the subject matter of the claims. The disclosures of all patents and publications cited herein are incorporated by reference in their entireties.

What is claimed is:

1. A catalyst for use in oxidative dehydrogenation processes comprising:
a dehydrogenative catalytically active component; and
an oxidative catalytically active component.

2. The catalyst of claim 1 wherein the catalyst has the general formula αAOₓ-PBO_{y}-γCO_{z}, wherein A is a precious metal and/or transition metal, B is a rare earth metal, C is an element chosen from Groups IIA, IIIA, and IVA, and O is oxygen.

3. The catalyst of claim 1 wherein the support comprises a plurality of discrete structures.

4. The catalyst of claim 3 wherein the discrete structures are particulates.

5. The catalyst of claim 4 wherein the plurality of discrete structures comprises at least one geometry chosen from the group consisting of powders, particles, granules, spheres, beads, pills, pellets, balls, noodles, cylinders, extrudates and trilobes.

6. The catalyst of claim 3 wherein at least a majority of the discrete structures each have a maximum characteristic length of less than 3 millimeters.

7. The catalyst of claim 6 wherein the majority of the discrete structures are generally spherical with a diameter of less than about 1 millimeter.

8. The catalyst of claim 6 wherein the majority of the discrete structures each have a characteristic length between 0.1 and 1 millimeter.

9. The catalyst of claim 1 wherein the catalyst comprises a mixed phase catalyst formed by the combination of an optimized dehydrogenative catalytically active component and an optimized oxidative catalytically active component.

10. The catalyst of claim 9 wherein the dehydrogenative catalytically active component and oxidative catalytically active component are combined through extrusion or compressing processes with bindery materials.

11. The catalyst of claim 9 wherein the dehydrogenative catalytically active component is selected from the group consisting of Rh, Ru, Pd, Pt, Au, Ag, Os, Ir, Sc, Ti, V, Cr, Mm, Fe, Co, Ni, Cu, Zn, Nb, Mo, Tc, Ru, Rh, Pd, Ag, Hf, Ta, W, Re and combinations thereof.

12. The catalyst of claim 9 wherein the dehydrogenative catalytically active component is selected from the group consisting of Pt, Au, Ag, Fe, Co, Ni, Mn, V or Mo and combinations thereof.

13. The catalyst of claim 11 wherein the oxidative catalytically active component is selected from the group consisting of La, Ce, Pr, Nd, Pm, Sm, Eu, Gd, Th, Dy, Ho, Er, Tm, Yb, Lu, Sc, Y, Th, V, Mn, Cr, Fe, Co, Sn, Mo, W, Cu, Ag and their respective oxides and combinations thereof.

14. The catalyst of claim 11 wherein the oxidative catalytically active component is selected from the group consisting of La, Yb, Pr, Sm, Ce, V, Cr, Cu, Sn and their respective oxides and combinations thereof;

15. The catalyst of claim 1 wherein the catalyst comprises a supported bifunctional catalyst formed by the combination of a supported dehydrogenative catalytically active metal and an oxidative catalytically active metal oxide.

16. A method for converting gaseous hydrocarbons to olefins comprising reacting a feed stream comprising an alkane with an oxidized bifunctional catalyst in a riser reactor to produce product vapors comprising olefins and paraffins and a reduced catalyst.

17. The method of claim 16, further including separating the reduced catalyst from the product vapors in a solid-gas separation vessel.

18. The method of claim 17 wherein the reduced catalyst and product vapors are separated by centrifugal force.

19. The method of claim 18 wherein the solid-gas separation vessel comprises a cyclone centrifuge.

20. The method of claim 17, further including separating the product vapors into an olefin stream and a paraffin stream in a gas-gas separation vessel.

21. The method of claim 20 wherein the product vapors are separated by boiling point differences.

22. The method of claim 21 wherein the gas-gas separation vessel comprises a distillation column.

23. The method of claim 20 wherein the paraffin stream is recycled back into the riser reactor.

24. The method of claim 17, further including regenerating the reduced catalyst with air or oxygen containing gas, in a regeneration reactor to form an oxidized catalyst.

25. The method of claim 24 wherein the oxidized catalyst is recycled back into the riser reactor.

26. The method of claim 16 wherein lattice oxygen and/or absorbed oxygen react with the feed stream.

27. The method of claim 16 wherein the reaction is adiabatic.

28. A method for the production of olefins comprising reacting a feed stream comprising an alkane with an oxidized bifunctional catalyst in a riser reactor to produce product vapors comprising olefins and paraffins and a reduced catalyst.
